(19) United States
(12) Patent Application Publication
(10) Pub. No.: US 2012/0082985 A1
(43) Pub. Date: Apr. 5, 2012

(54) Sensing And Identifying Biological Samples On Microfluidic Devices

(76) Inventors: Frederic Zenhausern, Fountain Hills, AZ (US); Ralf Lenigk, Chandler, AZ (US); Jianing Yang, Tampe, AZ (US); Zhi Cai, Chandler, AZ (US); Alan Nordquist, Payson, AZ (US); Stanley D. Smith, Phoenix, AZ (US); David Maggiano, Phoenix, AZ (US); Mrinalini Prasad, Chandler, AZ (US); Karem Linan, Queen Creek, AZ (US); Edward Olaya, Phoenix, AZ (US); Baiju Thomas, Chandler, AZ (US); Cedric Hurth, Tempe, AZ (US); Darryl Cox, Lavven, AZ (US); Mark Richard, Maricopa, AZ (US); Glen Mccarty, Tempe, AZ (US)

(21) Appl. No.: 12/672,889

(22) PCT Filed: Aug. 11, 2008
(86) PCT No.: PCT/US08/72819

§ 371 Date: Jun. 14, 2011

Related U.S. Application Data
(60) Provisional application No. 60/955,006, filed on Aug. 9, 2007.

(51) Int. Cl. C12Q 1/68; G01N 33/53; G01N 27/447; C12M 1/34; C12M 1/40

(52) U.S. Cl. 435/6.12; 435/6.1; 435/287.2; 204/452; 204/603

(57) ABSTRACT

A method, system, and apparatus for analysis of a biological sample includes receiving the sample, wherein the sample includes deoxyribonucleic acid (DNA), lysing the sample to obtain access to the DNA included in the sample, purifying the DNA in the sample to isolate the DNA from other components in the sample, amplifying the DNA, separating fragments of the amplified DNA, detecting the separated fragments using laser induced fluorescence, based on the detecting, generating a profile of the DNA in the received sample, comparing the generated profile with profiles of DNA stored in a database, and upon determining that the generated profile matches one of the stored profiles, identifying the source from which the stored profile was obtained, wherein the receiving, lysing, purifying, amplifying, and detecting are performed on corresponding portions of a microfluidic device, and wherein transporting the sample and the DNA to the portions of the microfluidic device and enabling the lysing, purifying, amplifying, separating, detecting, generating, comparing, and identifying are performed automatically without user interaction.

### CROSS-REFERENCE TO RELATED APPLICATIONS

This application claims the benefit of priority of U.S. Provisional Application Ser. No. 60/955,006 entitled “Sensing and Identifying Biological Samples on Microfluidic Devices,” filed on Aug. 9, 2007, the entire contents of which are incorporated herein by reference.

### TECHNICAL FIELD

This disclosure relates to analysis of biological samples, including samples containing biomolecules such as cells, proteins, nucleic acids, and preferably deoxyribonucleic acid (DNA).

### BACKGROUND

Nucleic acid, such as, DNA can be found in bodily fluids, such as saliva, blood and the like, as well as in other parts of the body including hair, skin, and the like. Identification based on DNA analysis includes collecting the biological samples containing DNA, processing the samples to obtain a profile of the DNA in the sample or fragments of said DNA and/or some by-products, and comparing the obtained profile against a reference profile. Biological samples that contain DNA can be found under controlled conditions, e.g., in a laboratory, and in uncontrolled environments, e.g., as forensic evidence in crime scenes. The individual processes of DNA analysis, including sample collection, sample processing, and sample detection, can be performed either on individual platforms or can be integrated onto a common platform. Miniature systems including microfluidic devices can be designed, fabricated, and configured to perform each process of DNA analysis. In addition, peripheral systems, including pumps, valves, and actuators, can be designed for use, in conjunction with the microfluidic device, to transport the biological samples from one domain to another on the microfluidic device, as well as to process the biological sample, by applying an electromagnetic wave such as heat, infra-red illumination and/or a mechanical force, for example pressure, and the like.

### SUMMARY

In one example, implementations of a method for integrated analysis of biological samples on microfluidic devices are described. The method can include receiving a biological sample, e.g., cells, containing DNA from a living organism, e.g., a human, automatically processing the sample to obtain access to the DNA in the sample, where the processing can include lysis and purification, amplifying the DNA preferably by enzymatic reactions, such as Polymerase Chain Reaction (PCR), electrophoretically separating some fragments of DNA or possibly other by-products, detecting the fragments using methods such as optical imaging such as laser induced fluorescence (LIF), gathering a profile of the fragments based on the detecting, and comparing the gathered profile with known profiles of DNA fragments to identify the source of the sample.

In one aspect, a method for analysis of a biological sample is described. The method includes receiving the sample at a sample collection chamber, wherein the sample includes deoxyribonucleic acid (DNA), automatically transporting the sample to a lysis unit, automatically operating the lysis unit to cause lysis to obtain access to the DNA, automatically transporting the DNA to a purification unit, automatically operating the purification unit to isolate the DNA from other components in the sample, automatically transporting the isolated DNA to an amplification unit, automatically operating the amplification unit to amplify the DNA, automatically transporting fragments of the amplified DNA to a separation unit, automatically operating the separation unit to separate the DNA into fragments, automatically operating a detection unit to detect the fragments using laser induced fluorescence, generating a profile of the DNA in the received sample based on the detecting, comparing the generated profile with profiles of DNA stored in a database, and upon determining that the generated profile matches one of the stored profiles, identifying the source from which the stored profile was obtained, wherein the sample collection chamber, the lysing unit, the purifying unit, the amplification unit, and the detection unit are formed in a substrate of a microfluidic device.

This, and other aspects, can include one or more of the following features. The sample can be automatically loaded in the sample collection chamber. Transporting the sample to the lysis unit can include transporting the sample via a micro-channel formed on the microfluidic device. Operating the lysis unit can include automatically transporting lysis reagents to the lysis unit, and automatically performing mechanical and electrical operations to cause lysis.

In another aspect, a preferred method for analysis of a biological sample is described. The method includes receiving the sample, wherein the sample includes deoxyribonucleic acid (DNA), lysing the sample to obtain access to the DNA included in the sample, purifying the DNA in the sample to isolate the DNA from other components in the sample, amplifying the DNA, separating fragments of the amplified DNA, detecting the separated fragments using laser induced fluorescence, based on the detecting, generating a profile of the DNA in the received sample, comparing the generated profile with profiles of DNA stored in a database, and upon determining that the generated profile matches one of the stored profiles, identifying the source from which the stored profile was obtained, wherein the receiving, lysing, purifying, amplifying, and detecting are performed on corresponding portions of a microfluidic device, and wherein transporting the sample and the DNA to the portions of the microfluidic device and enabling the lysing, purifying, amplifying, separating, detecting, generating, comparing, and identifying are performed automatically without user interaction.

This, and other aspects, can include one or more of the following features. Receiving the sample can include manually loading the sample. Receiving the sample can include automatically loading the sample. The microfluidic device can include one or more chambers to store reagents for the lysing, the purifying, the amplifying, the separating, and the detecting. Enabling the lysing, purifying, amplifying, separating, detecting, generating, comparing, and identifying are performed automatically without user interaction can include automatically transporting the stored reagents from the one or more chambers to the corresponding portions of the microfluidic device. The method can further include transporting the sample from one chamber to another chamber on the microfluidic device. The method can further include transporting the DNA from one chamber to another chamber on the microfluidic device. The method can further include transporting the DNA after amplifying the DNA and before separating fragments of the amplified DNA through a valve, the valve configured to remain in a closed state during the amplifying and switched to an open state during transporting the sample. The valve can be made using polymeric materials that can be formulated from a hybrid inorganic-organic material, e.g., an aerogel, a hydrogel, and the like. The DNA can be electrophoretically transported through the hydrogel valve. The sample can include a mixture of cells, e.g., sperm cells and epithelial cells as it is often collected in sex assault cases. The sperm cells and epithelial cells can be separated from one another. The separated sperm cells can be transported to a first chamber on the microfluidic device. The separated epithelial cells can be transported to a second chamber on the microfluidic device. The method can further include a data processing system with a graphical user interface presenting a display of control menus, wherein the one or more menus represent one or more corresponding controlling operations, the one or more operations comprising at least one of the lysing, the purifying, the amplifying, the generating, the comparing, and the identifying, and enabling a user to provide input to the operations through with the user interface. One of the one or more menus can be displayed when the corresponding operation is performed. A first menu corresponding to a first operation can be hidden and a second menu corresponding to a second operation can be displayed when the first operation ends and the second operation begins. A menu can display default operating conditions for operation. The default operating conditions can be altered based on user input. The input can include operating conditions.

In another aspect, a system for analysis of a biological sample is described. The system includes a sample collection chamber formed in a substrate, the sample collection chamber having an input port to receive a sample, wherein the sample includes deoxyribonucleic acid (DNA), a first transport mechanism configured to transport the sample from the sample collection chamber, a lysis unit, formed in the substrate, configured to obtain access to the DNA included in the sample received from the sample collection chamber, a second transport mechanism configured to transport the DNA from the lysis unit, a purification unit, formed in the substrate, configured to purify the DNA in the sample to isolate the DNA from other components in the sample received from the lysis unit, a third transport mechanism configured to transport the purified DNA from the purification unit, an amplification unit, formed in the substrate, configured to amplify the DNA received from the purification unit, a fourth transport mechanism configured to transport the amplified DNA, a separation unit, formed in the substrate, configured to separate fragments of the amplified DNA received from the amplification unit, a detection unit configured to detect the separated fragments using laser induced fluorescence, wherein a profile of the DNA in the received sample is generated based on the separated fragments detected by the detection unit, and a profile database configured to store profiles of DNA against which the generated profile is compared, wherein the source from which the stored profile was obtained is determined based on the comparing, and wherein the sample collection chamber, the lysis unit, the purification unit, the amplification unit, and the separation unit are configured to operate automatically without user interaction.

This, and other aspects, can include one or more of the following features. The sample can be manually loaded in the sample collection chamber. The microfluidic device includes peripheral devices incorporated in the substrate to enable operations performed in the sample collection chamber, the lysis unit, the purification unit, the amplification unit, and the separation unit and to enable transporting the sample and the DNA from one chamber on the microfluidic device to another chamber on the microfluidic device. The microfluidic device can include one or more chambers to store reagents used by the sample collection chamber, the lysis unit, the beautification unit, on the amplification unit, and the separation unit. Operating the lysis unit, the purification unit, the amplification unit, and the separation unit without user interaction can include transporting the sample and the DNA to corresponding units on the microfluidic device without user interaction, and transporting the stored reagents from the one or more chambers to the corresponding units without user interaction. The microfluidic device can include micro channels to transport the sampled to and from the sample collection chamber. The microfluidic device can include micro channels to crush both the DNA from one unit to another unit. The system can include a valve configured to enable transporting the DNA from the amplification unit to the separation unit, the valve configured to remain in a closed state computing the amplifying and switched to an open state during transporting the sample. The valve can be made of a hydrogel. The hydrogel can be positioned in a microchannel between the amplification unit and the separation unit. A potential difference can be applied across the microchannel to cause the movement of DNA from the amplification unit to the separation unit through the hydrogel. The sample can include sperm cells and epithelial cells. The sperm cells and epithelial cells can be separated from one another. The microfluidic device can include a first chamber into which the separated sperm cells can be transported. The microfluidic device can include a second chamber into which the separated epithelial cells can be transported.

In another aspect, a system for analysis of a biological sample is described. The system includes means for receiving the sample, wherein the sample includes deoxyribonucleic acid (DNA), means for automatically transporting the sample from the means for receiving, means for lysing the sample received from the means for receiving to obtain access to the DNA included in the sample, means for automatically transporting the sample from the means for lysing, means for purifying the DNA in the sample received from the means for lysing to isolate the DNA from other components in the sample, means for automatically transporting the sample from the means for purifying, means for amplifying the DNA received from the means for purifying, means for automatically transporting the amplified DNA from the means for amplifying, means for separating fragments of the amplified DNA received from the means for amplifying, means for detecting the separated fragments using laser induced fluorescence, means for generating a profile of the DNA in the received sample, based on the detecting, means for comparing the generated profile with profiles of DNA stored in a database, and means for identifying the source from which the stored profile was obtained, upon determining that the generated profile matches one of the stored profiles, wherein the means for receiving, means for lysing, means for purifying, means for amplifying, and means for detecting are formed in a substrate on a microfluidic device.

This, and other aspects, can include one or more of the following features. The system can include means for receiving the sample comprises means for manually loading the sample. The microfluidic device can include means for enabling operations performed in the means for receiving, means for lysing, means for purifying, means for amplifying, and means for separating and to enable transporting the sample and the DNA from one chamber on the microfluidic device to another chamber on the microfluidic device. The microfluidic device can include one or more means for storing reagents for the means for lysing, the means for purifying, the means for amplifying, the means for separating, and the means for detecting.

The methods, systems, and techniques described in this specification can present one or more of the following advantages. Multiple operations in the processing of a biological sample containing DNA, to identify the source of the sample can be performed on the same platform. The processes can be fully automated requiring no user interaction. Low concentrations of DNA in one or more different cell populations, e.g., sperm and epithelial cells, can be processed. Sperm cells can be automatically separated from epithelial cells, and DNA can be extracted automatically. All processes of analysis can be performed in a fully-contained cartridge platform which can eliminate the potential of contamination. The use of microfluidic techniques, such as acoustic mixing (e.g. cavitation microstreaming), in PCR and capillary electrophoresis can lead to shorter analysis times, e.g. sample input to profile output can take less than 2 hours). The compact design of the cartridge and instrumentation can enable the development of a portable STR typing system that can be deployed at a sample collection site, e.g., a crime scene.

The details of one or more implementations are set forth in the accompanying drawings and the description below. Other features and advantages will be apparent from the description and drawings, and from the claims.

### DESCRIPTION OF DRAWINGS

FIG. 1 is a schematic of an example of a system for automated analysis of a biological sample containing DNA.

FIG. 2 is a schematic of an example of a microfluidic device.

FIG. 3 is a schematic of an example of a microfluidic device.

FIGS. 4A and 4B are schematics of examples of a user interface to monitor the automated response system.

FIG. 5 is a flow chart of an example of a process for the automated integrated analysis of a sample containing DNA on a microfluidic device.

FIG. 6 is a schematic of an example of a microfluidic electrophoresis arrangement.

FIG. 7 is a schematic of an example of a laser induced fluorescence detection system.

FIG. 8 is a schematic of an example of a microfluidic device.

FIG. 9 is a schematic of an example of a printed circuit board.

FIG. 10 is a schematic of an example of a sensor module to enable microfluidic device operation.

FIGS. 11A and 11B are schematics of an example of a power supply to supply power to the automated response system.

Like reference symbols in the various drawings indicate like elements.

### DETAILED DESCRIPTION

DNA fingerprinting is a method for detecting and identifying a nucleic acid analyte (e.g. an unknown forensic sample). The method includes a comparison of the electrophoretic migration of restriction fragments of an unknown nucleic acid analyte to the electrophoretic migration pattern of a known genomic sample subjected to identical restriction treatment. This process is employed to identify or detect DNA. Variations on this process are known, including the use of specific hybridization probes to enhance accuracy by confirming that migration bands are homologous to specific nucleic acid sequences of interest. This is possible because restriction enzymes cleave DNA at specific loci, which will vary (i.e. exhibit polymorphism) with each genome. Thus, when DNA gel banding techniques were developed, it seemed possible that the technique would provide unique and unequivocal comparisons and identification between genomic samples.

DNA fingerprinting has been relied upon to analyze forensic evidence, for example, to obtain evidence of the identity of genetic material for criminal or paternity proceedings. The technique is also used to identify human remains or to determine relationship between species. DNA fingerprinting using VNTR loci complementary probes is useful not only in a forensic laboratory setting to provide individual identification and paternity testing, but also to investigate the taxonomic relationship among fish, birds, and other animals. In addition, it has been used for clarifying genetic relationship among related species, for discriminating pathogens from non-pathogens, and for determining the effect of environmental factors on evolutionary dynamics and speciation of microorganisms.

Accuracy has been improved over the years by applying some specific hybridization probes to electrophoresis patterns produced by DNA restriction fragments (Jeffreys et al, Nature, 314, 67-73, 1985). Such an approach led to the detection of polymorphisms in minisatellites DNA which can contain several repetitive sequences. The term “minisatellite” refers to any of a class of dispersed arrays of short (e.g. <50 bp) tandem direct repeat motifs that contain variants of a common core sequence. The majority of the minisatellites are distributed at the terminal ends of genomes, and these terminal repeats are often involved in the replication control of genes. The human-derived minisatellites are commonly used to prepare hybridization probes for forensic testing, for example, to provide individual identification and paternity testing. The multiloci human probes have been used for fingerprinting of DNA that can permit the construction of a population history and the geographical distance of different species.

Since the late 1980's, forensic DNA analysis has played a crucial role in the investigation and resolution of thousands of violent crimes. Currently, short tandem repeats (STRs) are the most widely used markers for forensic DNA testing. Because of their high discriminatory power, good resolution of alleles, and the ability to rapidly process samples using multiplexed polymerase chain reaction (PCR), 13 STRs have been chosen as the core loci upon which the FBI's National DNA Index System (NDIS) has been built. In recent years, other genetic polymorphisms, such as those found in the mitochondrial DNA (mtDNA) genome and the Y chromosome, have been shown to provide effective results that can augment traditional STR data. MtDNA analysis is especially useful for cases involving extremely degraded or limited biological residues, such as skeletal remains or shed hairs. Y chromosome markers can be beneficial in resolving sexual assault cases, particularly those with multiple male contributors. Single nucleotide polymorphisms, now known to be abundant throughout the nuclear genome, may become important genetic markers for the forensic scientist in the future.

The great variability of DNA polymorphisms has made it possible to offer strong support for concluding that DNA's from a suspect and from the crime scene are from the same person. Evidence that two DNA samples are from the same person is still probabilistic rather than certain. But with today's battery of genetic markers, the likelihood that two matching profiles came from the same person approaches certainty. STRs have small DNA sizes so their DNA can be amplified by PCR. This means that DNA from a trace sample, such as that from a cigarette or the saliva on a postage stamp, can be increased to an amount that can be readily analyzed. The interpretation of STRs is usually less ambiguous than that of VNTRs and the process is more rapid, e.g., days instead of weeks. It also lends itself to automation, and kits are now available in which 16 loci can be analyzed simultaneously. In the USA, the Federal Bureau of Investigation (FBI) has chosen 13 STR loci to serve as core loci for the Combined DNA Index System (CODIS), the intention being that all forensic laboratories be equipped to handle these 13. CODIS is a national database and searching mechanism, which now utilizes these 13 core STR loci. Today, more than 170 laboratories have now installed the CODIS system and more than 3,000,000 STR profiles from convicted felons are already on file.

In order to address the growing demand for DNA fingerprinting and so the huge backlog of samples, there is a critical need for improvements in collection and purification techniques. The forensic DNA community would therefore greatly benefit from a DNA technology platform suitable for automated high-throughput processes that can be highly reliable and accurate for forensic use (DOJ/NIJ report: The Future of DNA Testing, 2000). Such DNA technology will result in faster, more robust, more informative, less costly and/or less labor intensive identification, collection, preservation, and/or analysis of DNA evidence at crime scenes. During the last few years, several research groups have reported, in the literature, the development of miniaturized system for analyzing STRs. For example, Ehrlich et al. (Schmalzing et al. 1998, 1999) have reported a laser-induced fluorescence detection system that can do a quick analysis of eight of the CORE loci. With a resolution of four bases, the process can be completed in 2 minutes. Resolution to one base requires about 10 minutes. The device is about 150 mm in diameter and made from fused-silica wafers. The leading approaches employ photolithography and chemical etching techniques to manufacture microchannels in glass or silicon substrates. Miniaturized system capable of eletrophoresing and capturing STR data are also available, with some recent advances consisting of a glass microfluidic device for amplification and separation of the STR fragments (R. Mathies et al., Integrated portable microchip system for rapid forensic STR typing, Proceedings 17th Int. Symp. On Human Identification, Oct. 9-12, 2006; P. Liu et al, Anal. Chem., 79, 1881-1889, 2007). These systems do not allow for direct sample preparation from body fluids as they do not consist of sample extraction and differential cell lysis functionalities and they are completed by attaching a discrete silicon amplification module to an analysis capillary electrophoresis glass chip.

The disclosure relates to a method and apparatus that allow for performing automated continuous flow processes of sexual assault forensic samples with moderate complexity. In particular, the apparatus is configured to perform multiple preparation processes consisting of a differential cell extraction from a mixture of biological specimens from a forensic evidence sample, amplify some genetic materials of the cells and perform a simultaneous detection of male and female DNA by detecting the respective STR profiles that can then be processed into a searchable sample database. All processing steps can be performed onto an all-polymer monolithic cartridge containing all fluidic actuators (e.g. reservoirs, channels, valves, pumps), electronic sensors (e.g. heater elements, temperature diodes, electrodes) and, electrical and optical interfaces. The capability of using polymeric substrate for fabricating small electrophoretic separation channels allows ranging the electro-osmotic flow (EOF) from high value to no EOF when using smooth polymeric walls that can be preferably formed by injection molding processing.

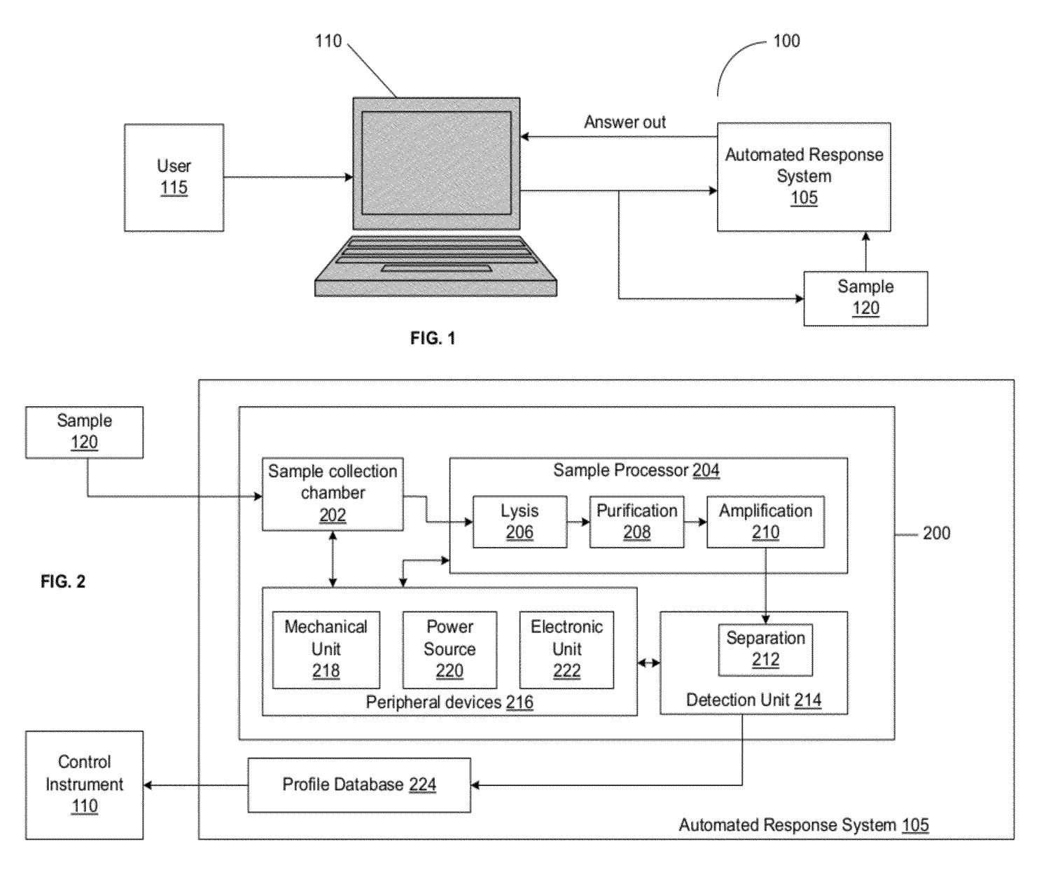

FIG. 1 depicts an example of a system 100 for analysis of a biological sample containing DNA. The system 100 includes an automated response system (ARS) 105, which includes a microfluidic device and peripheral devices. The ARS 105 can be configured to perform all the steps of sample analysis. Further, the system 100 can include a control instrument 110, e.g., a computer, to provide input to and receive output from the ARS 105. A user 115 can interact with the ARS 105 by providing instructions to operate the ARS 105 through the control instrument 110. The sample 120 can be introduced into the ARS 105 either manually by the user 115 or automatically by input provided to the control instrument 110 by the user 115.

In some implementations, a user 115 can collect a sample 120 containing analytes of interest, e.g., DNA. The user 115 can introduce the sample to the ARS 105 either manually, e.g., by pipetting the sample into one or more appropriate chambers in the ARS 105, or automatically, e.g., by providing instructions, using the control instrument 110, to a device configured to transport the sample 120 from a repository to the ARS 105. The ARS 105 is configured to receive the sample, process the sample, and obtain a profile of the DNA contained in the sample. Further, the ARS 105 is configured to compare the profile obtained from the sample with profiles obtained from known sources to identify the source from which the sample was obtained. Since DNA profile is unique to each source, using the DNA profile, the source of the sample can be identified. The control instrument 110 provides input to the ARS 105 and to the peripheral devices that manipulate the sample on the microfluidic device. In some implementations, the ARS 105 can be controlled using LabVIEW Software offered by National Instruments Corporation (Austin, Tex., USA). A user interface can be created using the LabVIEW software that can include illustrations of the one or more components of the ARS 105. The user interface can be designed such that operations being performed on the ARS 105 can be represented in the user interface to enable a user to monitor the integrated analysis. Alternatively, the ARS 105 can be controlled using any appropriate software. Further, the ARS 105 provides output to a display device of the control instrument 110. In some implementations, once the user 115 loads the sample into the microfluidic device in the ARS 105, the control instrument 110 can automatically control the ARS 105 to process the sample. In other implementations, a user 115, upon viewing the output provided by the ARS 105, can provide additional instructions to process the sample 120 to the control instrument 110. In this manner, interaction of a user 115 with the ARS 105 can be minimized and, in some instances, eliminated.

FIG. 2 depicts an example of a microfluidic device 200 configured to analyze a biological sample to identify a profile of the DNA in the sample. The microfluidic device 200 includes a sample collection chamber 202 configured to receive the sample 120. In some implementations, the sample collection chamber 202 can occupy a volume of 400 μl. Alternatively, the sample collection chamber 202 can be designed to occupy any volume based on the sample being analyzed. In some implementations, a user 115 can manually load the sample 120 into the sample collection chamber 202. For example, the sample 115 can be a combination of epithelial cells and sperm cells. The cells can be collected using collection devices, e.g., a cotton swab, and, subsequently, eluted using appropriate elution buffer. Following elution, a known volume, e.g., 300 μl, of the sample 115 in the elution buffer can be injected into the sample collection chamber using a pipette. After injection, the epithelial cells of a mixed sample can then be lysed using a lysis buffer, e.g., a composition containing 320 μl Tris/EDTA/NaCl, 20 μl 20% Sarkosyl, 60 μl H2O, and 1 μl proteinase K, that can be filled into the cartridge during the design process. In other implementations, the user 115 can provide instructions to the control instrument 110 to automatically load the sample 120 into the sample collection chamber 202. For example, peripheral devices 216, such as micro-valves and micro-pumps, can be incorporated in the design of the microfluidic device of the ARS 105. The micro-valve can include a polymeric material deposited into a cavity fabricated in the microfluidic device. The peripheral devices 216 can include a backplane heating element, e.g., a ceramic resistive component arranged on an electronic circuit of a printed circuit board, thermally resistive carbon ink positioned on the back surface of the sample collection chamber 202, and the like, that can be used to control the micro-valves and the micro-pumps to automatically load the sample 120 into the sample collection chamber 202. In some implementations, the peripheral devices 216 can be operated such that heat from the devices 216 can cause the polymeric material in the micro-valves and the micro-pumps to melt, enabling sample transport.

The microfluidic device 200 includes a sample processor 204 configured to perform operations including enabling accessing the DNA in the sample 115, purifying the accessed DNA, and amplifying the DNA using the PCR process. In some implementations, the sample processor 204 includes a lysis chamber 206 in which the sample 120 can be processed to access the DNA in the sample 120. In some implementations, the sample 120 can be a cell where the DNA is encapsulated in a cell membrane in addition to other cellular components. When the ARS 105 transports the sample 120 in elution buffer into the lysis chamber 206, the ARS 105 can expose the sample 120 to one or more lysis buffers that can break open the cell membrane, thereby allowing access to the encapsulated DNA.

The sample processor 204 includes a purification chamber 208 in which the sample 120 is purified by separating the DNA from the other components in the sample. In implementations where the sample 120 is a cell, the components of the sample 120 other than the DNA may inhibit downstream processes, such as DNA amplification. Therefore, it may be necessary to extract and purify the DNA and remove the other components prior to further processing. In some implementations, the ARS 105 transports the lysed sample, including the DNA, to the purification chamber 208. The ARS 105 can perform DNA purification in the purification chamber 208 using magnetic beads coated with a material suitable to capture DNA. In some implementations, commercially available ChargeSwitch® beads, offered by Invitrogen™ (Carlsbad, Calif.) can used for DNA capture. Alternatively, or in addition, beads of other sizes can be used, where the beads can include various oligonucleotide immobilization strategies to link covalently macromolecular receptor(s) such as nucleic acids to a magnetic surface or using a combination of a silica-based surface with a high magnetic susceptibility materials for efficient coupling procedure. In some implementations, robust aptamer based coupling using amide bond to couple the DNA to macroporous carboxylic acid-based magnetic materials can be used for an oligonucleotidic selector binding. The purification can be based on solid phase extraction principles. The DNA in the sample 115 can bind to the material on the surface of the magnetic beads under optimal conditions of pH, temperature, and the like. The conditions can be manipulated such that other components in the sample 120 do not bind to the magnetic beads. The unbound components can be removed from the purification chamber 208. Subsequently, the conditions inside the purification chamber 208 can be altered to release the DNA from the surface of the magnetic beads. In some implementations, the DNA can be caused to bind to the magnetic beads by subjecting the DNA and the beads to an environment where the pH is less than 6.5. Subsequently, the DNA can be released from the beads by changing the pH of the environment to or greater than 8.0. In this manner, the DNA binding and releasing from the magnetic beads can be controlled by varying the pH of the environment. In some implementations, the reagents used for DNA extraction and purification can be obtained from commercially available kits, e.g., kits offered by DYNAL®, Invitrogen (Carlsbad, Calif., USA), Agencourt Bioscience Corporation (Beverly, Mass., USA), and the like.

The microfluidic device 200 includes an amplification chamber 210 where the extracted and purified DNA samples can be amplified by PCR. Typically, the total volume for the STR-typing multiplex PCR can range from 10-25 μl. The standard PCR conditions and volumes, e.g., 25 μl can be used for the integrated device. The ARS 105 can transport the extracted and purified DNA from the purification chamber 208 to the amplification chamber 210 and further expose the DNA to conditions suitable for DNA amplification by PCR.

The microfluidic device 200 includes a separation unit 212 designed for the electrophoretic separation of the amplified DNA fragments. The ARS 105 can transport the amplified DNA to the separation unit 212 for electrophoretic separation. In some implementations, a porous material, e.g., hydrogel, can be positioned between the sample processor 204 and the separation unit 212 to serve as a valve. The hydrogel valve can be designed such that, in the closed state, the hydrogel valve can withstand any pressure built during the PCR process, thereby preventing the sample from being transported to the separation unit 212 prior to completion of PCR. In the open state, the hydrogel valve can enable sample transport to the separation unit 212. For example, in the open state, the sample can be electrophoretically be transported through the hydrogel layer in the hydrogel valve from the amplification unit 210 to the separation unit 212. In addition to enabling sample transport, the hydrogel can cause a separation of the DNA from the PCR medium. In addition, the hydrogel can cause a stacking effect, where the DNA in the sample can be concentrated in a plug of sample by establishing a buffer concentration gradient between the PCR chamber and the gel. The hydrogel can be positioned between the amplification unit 210 and the separation unit 212 during the design stages of the microfluidic device. The composition of the hydrogel can be chosen such that the hydrogel valves can withstand pressures built in the PCR chamber, e.g., 10 psi. In addition, the voltages applied across the hydrogel valve can be chosen based on the electrophoretic mobilities of the DNA and the components of the PCR media, such that only the DNA is transported into the separation unit 212. This separation of DNA and PCR media can further be improved by choosing hydrogels where the cross-linking in the gels enable transport of only DNA and not the PCR media. The separation chamber 212 can be included in a detection unit 214 which can be combined with the microfluidic device 200. The detection unit 214 can be configured to detect the profile obtained from DNA fragments by methods such as LIF.

The microfluidic device 200 includes peripheral devices 216 including mechanical units 218, power sources 220, and electronic units 222. The peripheral devices 216 enable transporting the sample 120 from one portion of the microfluidic device to another through the microfluidic channels. The mechanical units 218 can include valves, pumps, actuators, and the like. The power source 220 can operate one or more energy sources, including mechanical energy sources, e.g., mixers, heat energy sources, e.g., heaters, voltage sources, and the like. The electronic units 222 can be configured to provide voltage input to the power sources 220 and to sense the voltage output from the microfluidic device and peripheral devices. The peripheral devices 216 can be controlled by a user 115 through the control instrument 110. In some implementations, the microfluidic device 200 and the peripheral devices can be integrated onto the same platform. Reagents, such as lysis buffers, purification buffers, wash buffers, electrophoresis reagents, and the like, can be filled into reservoirs on the microfluidic device 200 prior to introducing the sample. The ARS 105 can control the peripheral devices 216 to make the reagents available for sample processing by transporting the reagents to appropriate portions of the microfluidic device 200. In addition, the ARS 105 can control the peripheral devices to apply heat, pressure, and the like, to the sample and the reagents to facilitate sample processing. In some implementations, the control instrument 110 can be configured to manipulate the components of the peripheral devices 216 to process the sample 120 with no or minimum input from a user 115. Alternatively, the peripheral devices 216 can transmit information obtained from the ARS 105 to the control instrument 110. In response to the transmitted information, the ARS 105 can receive further instructions to process the sample. The ARS 105 can process the sample based on the received instructions using the peripheral devices. Alternatively, or in addition, the ARS 105 can include a printed circuit board (PCB) on which the one or more portions of the mechanical unit 218, the power source 220, and/or the electronic unit 222 can be incorporated. The units of the peripheral devices 216 incorporated onto the PCB can operate valves, pumps, actuators, and the like that are designed into the microfluidic device 200 structure. The PCB and the microfluidic device 200 can be designed such that the peripheral devices 216 on the PCB align with the corresponding microfluidic device 200 architecture. Further, the ARS 105 can be configured such that the microfluidic device 200 and the PCB can be positioned in the ARS 105. Such configurations can include incorporating jigs and/or fixtures in the ARS 105 design to facilitate positioning and aligning of the microfluidic device 200 and/or the PCB. The DNA profile obtained from a sample can be stored in a profile database 224. The ARS 105 can include a profile database 234, in which, the profile can be stored as a reference profile if the sample is obtained from a reference. The ARS 105 can populate the profile database by collecting reference DNA profiles from multiple references. If the sample is a target sample, where the source of the sample is unknown, then the ARS 105 can store the target sample DNA profile in the profile database 224. The control instrument 110 can be configured to compare the reference profiles and the target sample DNA profile. If the target sample DNA profile matches one of the reference profiles, then the control instrument 110 can present the reference as the source of the target sample. In some implementations, the profile database 224 can be part of the ARS 105. In other implementations, the profile database 224 can be part of the control instrument 110. In other implementations, the profile database 224 can be external to both the ARS 105 and the control instrument 110 and can be accessed through wired or wireless methods.

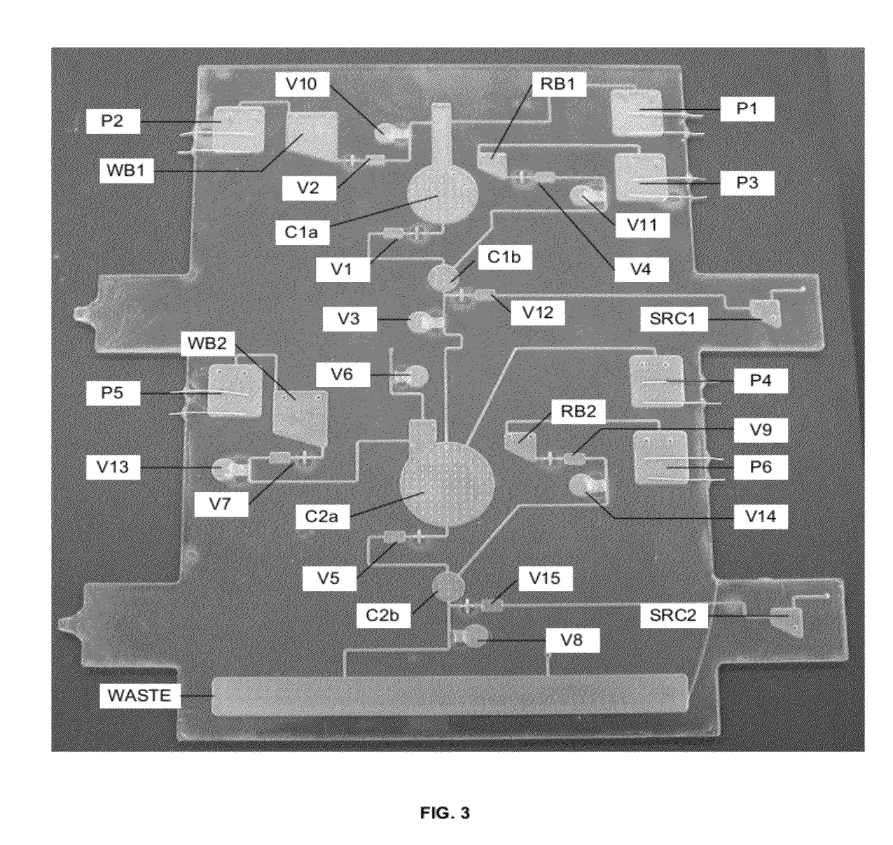

FIG. 3 depicts a schematic of the microfluidic device including peripheral components for preparing a sample to identify the source of the sample based on a profile of DNA obtained from the sample. The sample is prepared by purifying the DNA in the sample and amplifying the DNA. The microfluidic device includes peripheral devices 216 which, in turn, includes mechanical units 218, power sources 220, and electronic units 222. In addition, the microfluidic device 200 can also include reservoirs to store various buffers, e.g., lysis buffer, purification buffer, PCR buffer, separation buffer, and the like. The ARS 105 can manipulate the peripheral devices 216, based on user input 115, to transport the buffers to different chambers on the microfluidic device 200. The user can load a sample into reservoir C1a, where the sample can be a combination of epithelial cells and sperm cells. A known volume, e.g., 8.48 μl, of lysis buffer, e.g., sperm lysis buffer when the sample 120 is sperm can be loaded into reservoir C2a by the user 115. One example of a suitable lysis buffer for the disruption of sperm cell membranes is composed 150 μl Tris/EDTA/NaCl, 50 μl 20% Sarkosyl, 7 μl 1M DTT, 150 μl H₂O, and 2 μl proteinase K. The volume of C2a is determined by the amount of sample plus epithelial cell lysis buffer in addition to the wash buffer used to rinse the magnetic beads that capture epithelial cell DNA. Subsequent to these two procedures performed by the user 115, the microfluidic device 200 can be placed into the ARS 105.

The ARS 105 can incubate the sample at a pre-determined temperature, e.g., 37° C., for a pre-determined time, e.g., 1 hour. The ARS 105 can control the temperature using, e.g., a RTD (Resistance Temperature Detector) diode on the PCB behind reservoir C2a using a heater system that forms a feed-back loop with the diode and the heater to ensure precise temperature control. Alternatively, any other thermal circuitry with an active thermal feedback can be used for temperature control. The ARS 105 can also incubate the lysis buffer in reservoir C2a for a pre-determined period, e.g., 2 hours, at a pre-determined temperature, e.g., 37° C. The ARS 105 can transport the remaining sample after the capture of epithelial cell DNA from reservoir C1a to the sperm lysis buffer in reservoir C2a to lyse the sperm cells present in a sample. The sperm cells are being transported in the epithelial cell lysis buffer. In some implementations, the sperm cell lysis buffer concentration can be optimized to account for any dilution. In both chamber C1a and C2a the cell-lysis and DNA-binding takes place simultaneously; the magnetic bead solution is mixed with lysis buffer. In some implementations, the ARS 105 can transport the lysis buffer and/or the sample between the reservoirs by operating electrolytic micropumps and valves incorporated into the microfluidic device 200. In some implementations, single-use heat actuated polymer on/off and off/on valves can be used. Alternatively, or in addition, other valve types (duck-bill valves, ball valves) can also be used. The reagents included in the lysis buffer in reservoir C2a can lyse the sample and release the DNA. The released DNA is captured by the magnetic beads, where the capture is facilitated by mixing. The ARS 105 can mix the sample with the magnetic beads at regular intervals, using the PZT mixing process. In addition, the ARS 105 can include a linear solenoid that drives a magnet attached to the PCB behind the reservoir C1a. The ARS 105 can use the magnetic field to keep the magnetic beads in a state of agitation to prevent settling due to gravity. Subsequent to enabling DNA capture on the magnetic bead bed, the ARS 105 can wash the beads and elute the DNA from the magnetic beads using a release buffer. In some implementations, the ARS 105 can transfer the eluted DNA to sample recovery chamber 1 (SRC1). The eluted DNA is available for transport amplification by PCR. In other implementations, the ARS 105 can make available the eluted DNA, to a user, for retrieval and transfer to an external amplification unit.

The ARS 105 can transport the eluted DNA to the amplification unit 210 where a reservoir (PCRC1) contains a PCR multiplex mixture. In some implementations, the multiplex mixture can be obtained as part of the AmpF/STR® Identifiler® PCR Amplification Kit offered by Applied Biosystems (Foster City, Calif.). The ARS 105 can transport the products of the PCR from the amplification unit 210 to the separation unit 212 for separation by capillary electrophoresis. In some implementations, the PCR products can be transported electrophoretically, e.g., through a hydrogel valve, from the amplification unit 210 to the separation unit 212. Alternatively, or in addition, the PCR products can be transported by electro-osmotic mechanisms and/or pressure-driven flow mechanisms.

Upon transporting the PCR products to the separation unit 212, the ARS 105 can mix the PCR products with formamide and internal size standard (DNA ladder). The formamide and the internal size standard can be pre-loaded onto the microfluidic device 200. In some implementations, the separation unit 212 can include a polymeric microchannel hot-embossed using a reusable silicon master, for example, a 1.1 mm thick, 10 cm×2 cm polymer coupon, in particular plastic cyclic olefin copolymer (COC) coupon. The dimensions of the channel can be pre-determined, e.g., approximately 9 cm in total length, where the detection is made at approximately 6.2 cm from the injection point, width at the top of the microchannel approximately 60 μm, width at the bottom of the microchannel approximately 39 μm, a channel depth of 25 μm, and a cross section of approximately 1237.50 μm². The ARS 105 can load a polymer matrix, e.g., a linear polyacrylamide gel optimized for DNA separation efficiency, such as Performance-Optimized Polymer (POP-5) offered by Applied Biosystems (Foster City, Calif., USA) into the COC separation channel for a pre-determined time, e.g., 30 minutes, at a pre-determined temperature, e.g., room temperature. The ARS 105 can mix sample with size standard, such as LIZ® offered by Applied Biosystems in a known volumetric ratio, e.g., 1:1 and, further, with a known volume of formamide, e.g., Hi-Di™ formamide offered by Applied Biosystems. The remaining microchannels of the separation unit 212 can be filled with a buffer, e.g., ACE buffer (offered by Amresco, Inc., Solon, Ohio), to prevent the polymer matrix filled channels from drying out.

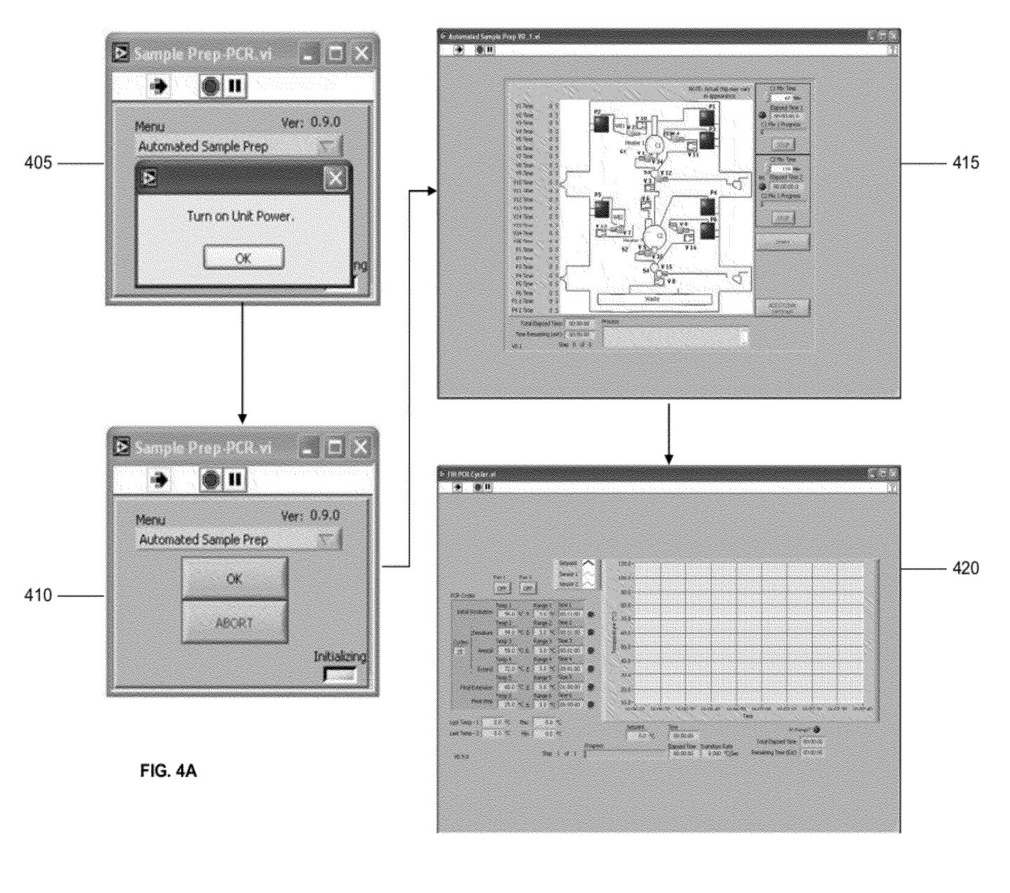

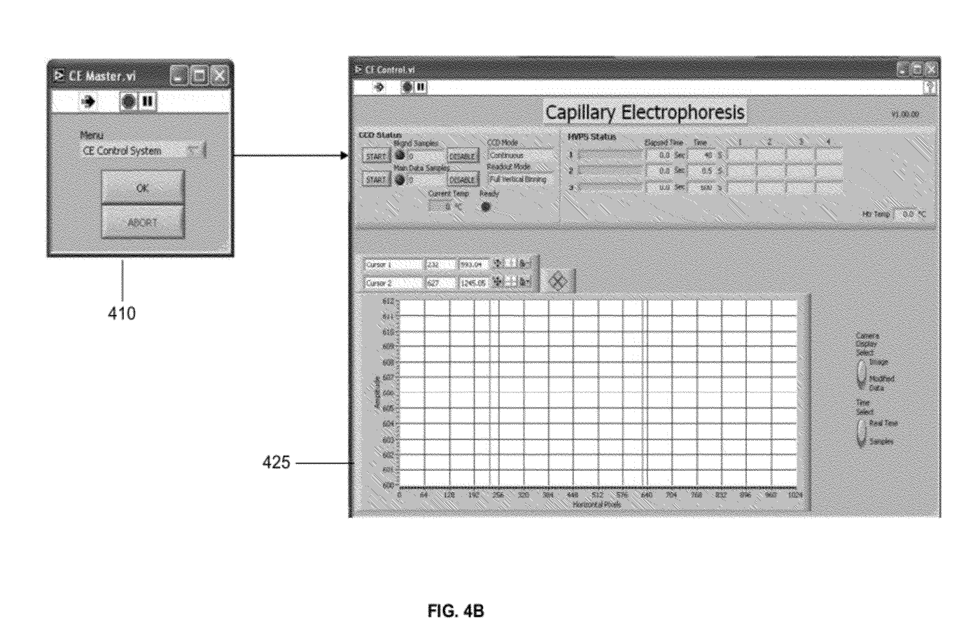

FIGS. 4A and 4B are schematics of user interfaces (UIs) to monitor the operations of the ARS 105. In some implementations, the user interfaces can be created using software applications, e.g., LabVIEW. The control instrument 110 can be configured to display the UIs on a display device operatively coupled to the control instrument 110. In addition, the control instrument 110 can also be operatively coupled with an input device, e.g., key board, a mouse, or both, to receive input from a user. The UIs can include one or more screens, displaying menus, that can either be displayed individually or simultaneously on the display device. For example, FIG. 4A depicts a status screen 405 indicating the status of the system 100 displayed when a user launches the application to operate the ARS 105 on the control instrument 110. Alternatively, the status screen 405 can be displayed when a user powers the control instrument 110. The control instrument 110 can detect the status of the ARS 105, e.g., whether the ARS is turned on or is off, and, if the ARS 105 is off, then display, on the status screen 405, a message prompting the user to turn on the ARS 105. Subsequently, the user can be displayed an operation screen 410 using which the user can initiate sample preparation by choosing the “Automated Sample Prep” option from a drop-down menu and selecting “OK.” In response, the ARS 105 can perform the lysis 206 and purification 208 steps. Once the “Automated Sample Prep” process begins, the display device can display an operation screen 415, which can display information including a schematic of the ARS 105, a list of valves, actuators, and other components of the ARS 105, the operation time of each component, and the like. Upon completing the lysis 206 and purification 208 steps, the display device can display a PCR screen 420, which can direct the user to commence PCR. In addition, the PCR screen 420 can receive input from the user to control the operating conditions of the PCR, e.g., temperature, time, and the like. The control instrument 110 can use the input provided to the UI to control the PCR process on the ARS 105. In some implementations, the PCR screen 420 can display the default PCR operating conditions and the ARS 105 can perform PCR under the default conditions in the absence of input from the user. Alternatively, a user can over-write the default operating conditions with the user's operating conditions.

FIG. 4B depicts a UI displaying the status screen 410 after completion of PCR steps 210. The user can select the separation step 212 by selecting “CE Control System” from the drop down box and selecting “OK.” In response to the user selection, a CE screen 425 can be displayed on the display device. The CE screen 425 can be configured to display the status of the separation steps including the status of the high voltage power supply (HVPS) designed and operatively coupled to the ARS 105 to perform electrophoretic separation, time durations of experiments, outputs of the detection unit 214, and the like. In addition, the CE screen 425 can also be configured to display the different stages of the electrophoretic separation process, e.g., the injection stage, the separation stage, and the like. The CE screen 425 can also display the voltage, time settings, progress at each stage, and the like, and can also be coupled with temperature and voltage monitors operatively coupled to the ARS 105. In addition, the user can provide input to the control instrument 110 using the CE screen 425 to save the separation data to a storage device. In some implementations, the CE screen 425 can display the default storage location of the data. A user can provide input to the CE screen 425 to alter the storage location. In some implementations, the display device can display one screen at a time and enable a user to access different screens and/or to abort the operations represented by the displayed screen, e.g., by displaying selectable buttons on the UI to switch between screens. In other implementations, all screens of the UI can be displayed simultaneously on the display device. In addition, a screen corresponding to an operation being performed on the ARS 105 can be highlighted while other screens can be de-activated. The UI can include an “Abort” button that a user can select to abort the operations being performed on the ARS 105.

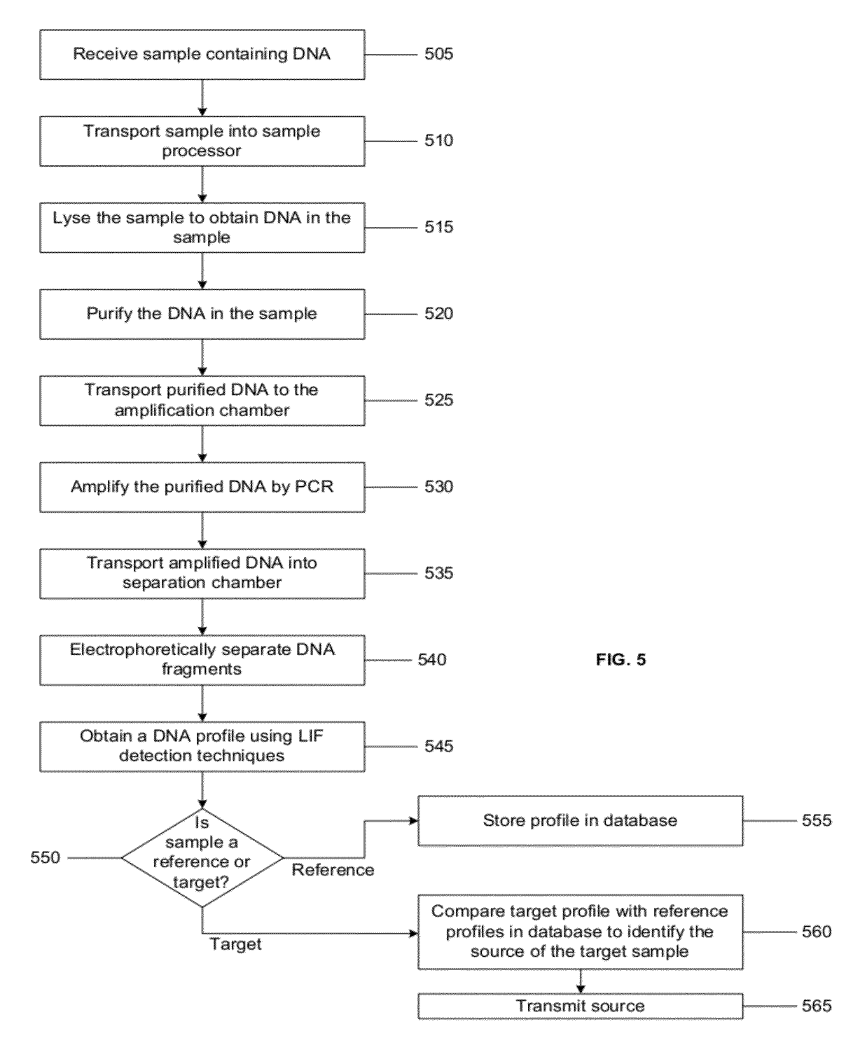

FIG. 5 is a flow chart of an example of a process for fully automated integrated analysis of a sample containing DNA on a microfluidic device to identify the source of the DNA. The sample (e.g., blood, saliva, and the like) can be either a reference sample or a target sample and can be received at 505. In some implementations, the sample can be received in a reservoir on the microfluidic device from a user who manually provides the sample. In other implementations, the sample can be automatically received by a user controlled instrument configured to provide the sample. The sample can be transported through microchannels in the microfluidic device to the sample processor, also fabricated into the microfluidic device, at 510. The sample can be transported using actuators, e.g., pumps, and the flow can be manipulated using valves. The sample containing DNA can be lysed to obtain the DNA in the sample 515. In some implementations, the sample can be transported into a chamber on the microfluidic device where the lysis buffer is stored. In other implementations, the lysis buffer can be loaded onto the microfluidic device prior to commencement of sample analysis. The sample and the lysis buffer can be transported to a lysis unit through microchannels where the sample can be lysed in the presence of the lysis buffer. Lysis breaks open the sample allowing access to the DNA contained in the sample.

Subsequent to lysis, the DNA in the sample can be purified at 520. Purification can include separating the DNA from other components of the sample. In some implementations, the lysis and purification can occur in the same chamber of the microfluidic device. In other implementations, the sample can be transported to a purification chamber to separate the DNA from other components of the sample. In some implementations, the DNA can be separated from the sample by solid phase extraction principles where the purification chamber can include a medium (e.g., magnetic beads coated with a suitable material) onto which the DNA can be bound, under appropriate experimental conditions (e.g., pH, temperature, and the like) while the other unbound components of the sample can be washed away. Subsequently, the bound DNA can be released by flowing a wash buffer over the medium to which the DNA is bound under different experimental conditions. In other implementations, the unwanted components of the sample can be bound to a phase while the DNA can be washed into a separate chamber on the microfluidic device. In some implementations, in addition to separating the DNA from the sample, the purifying can enrich the concentration of DNA by collecting DNA spread over a larger into a concentrated volume of DNA. Following purification, the purified DNA can be transported to the amplification unit at 525. The amplification unit can include reservoirs, heaters, and the like, and the transporting can be done via microchannels fabricated in the microfluidic device.

The DNA can be amplified by PCR at 530. The amplified DNA can be transported into the separation chamber at 535. Fragments of the amplified DNA can be separated by electrophoresis at 540. The amplified DNA can be mixed with formamide and internal size standard (DNA ladder) that can be pre-loaded onto reservoirs in the microfluidic device. The DNA can be migrated along a DC electrical field, e.g., 180 V/cm, through a polymeric sieving matrix, e.g., polyacrylamide gel pressure-loaded into a 50 μm wide, 20 μm deep, and 8 cm long semi-elliptic microchannel. A DNA profile using laser induced fluorescence (LIF) detection techniques can be obtained at 545. The migrating DNA, mixed with a fluorescence-based detection dye, to form a DNA-dye complex and the DNA-dye complex can be detected using an LIF detector. The LIF detector can include an argon-ion laser (488 nm and 514 nm wavelength lines) which can be focused within a micro-channel of the microfluidic device. In some implementations, the micro-channel, where the laser beam is focused, can be between 40 μm and 50 μm in diameter. A microscope objective with high numerical aperture, e.g., Olympus, LUCPLN FLN, 20×, NA=0.45, can be used. A dichroic mirror, or beam splitter, e.g., Chroma Z525RDC) can be used to direct the reflected light onto the sample via the objective and prevent the laser light reflected by the sample from further propagating into the detector. The dichroic mirror will allow the fluorescent signal obtained from the DNA-dye complex to be transmitted.

A check can be performed to determine if the sample is a reference sample or a target sample at 550. If the sample is a reference sample, then the obtained DNA profile is stored in a database at 555. If the sample is a target sample, then the obtained DNA profile is compared with profiles in the database obtained from reference samples at 560. Since the DNA profiles are unique to each source, if the profile obtained from the target sample matches one of the profiles obtained from reference samples, then the source of the target DNA can be identified. The identified source can be transmitted to a control instrument at 565. Alternatively, the identified source can be transmitted to any device over any network, e.g., wired, wireless, and the like. The steps 510-565 can be performed with no user interaction. At each step, feedback signals can be presented to a control instrument, which a user can use to monitor the analysis. Since the received sample, for example, is a sample collected at a crime scene, and since the obtained profile identifies the source from which the sample originated, the automated integrated DNA analysis system represents a “sample in/answer out” type of system.

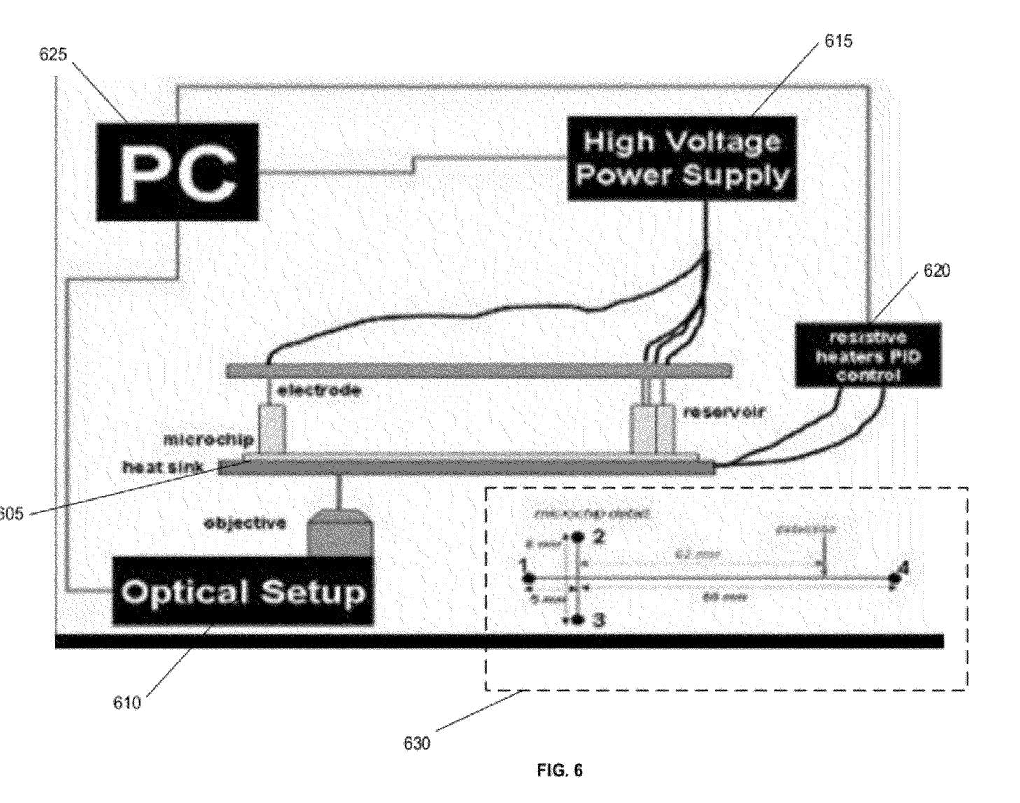

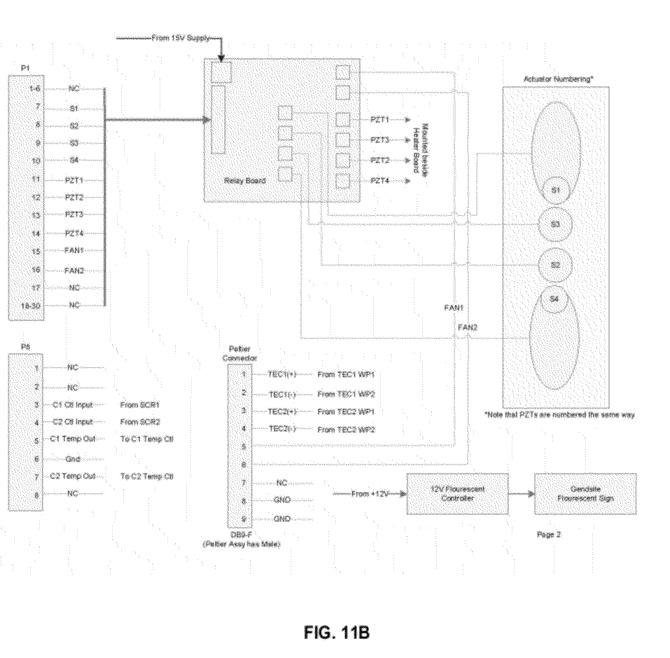

FIG. 6 depicts a schematic of an example of a microfluidic capillary electrophoresis arrangement. The microfluidic device 610 can include chambers into which one or more electrodes can be inserted to apply a potential difference across a micro-channel in the microfluidic device. In addition, the microfluidic device can include one or more reservoirs into which buffers can be filled. Additionally, probes to monitor the operating conditions in the microfluidic device, e.g., voltage, current, and the like, can be placed in the reservoirs. The arrangement can include an optical set up 610 which can be an LIF detection system configured to excite the DNA-dye complex and detect fluorescence emitted by the complex. The arrangement can include a high voltage power supply (HVPS) 615 configured to provide the voltages required to transport the DNA across the micro-channels of the microfluidic device. An example HVPS 615 used in the arrangement is described with reference to FIGS. 11A and 11B. The arrangement can further include resistive heaters PID control 620 to control and monitor the temperatures in the microfluidic device. The optical set up 610, the HVPS 615, and the heaters PID control 620 can be controlled either independently or using a computer (PC) 625. The arrangement further illustrates the design of the micro-channels in the microfluidic device 630 for performing microfluidic electrophoresis.

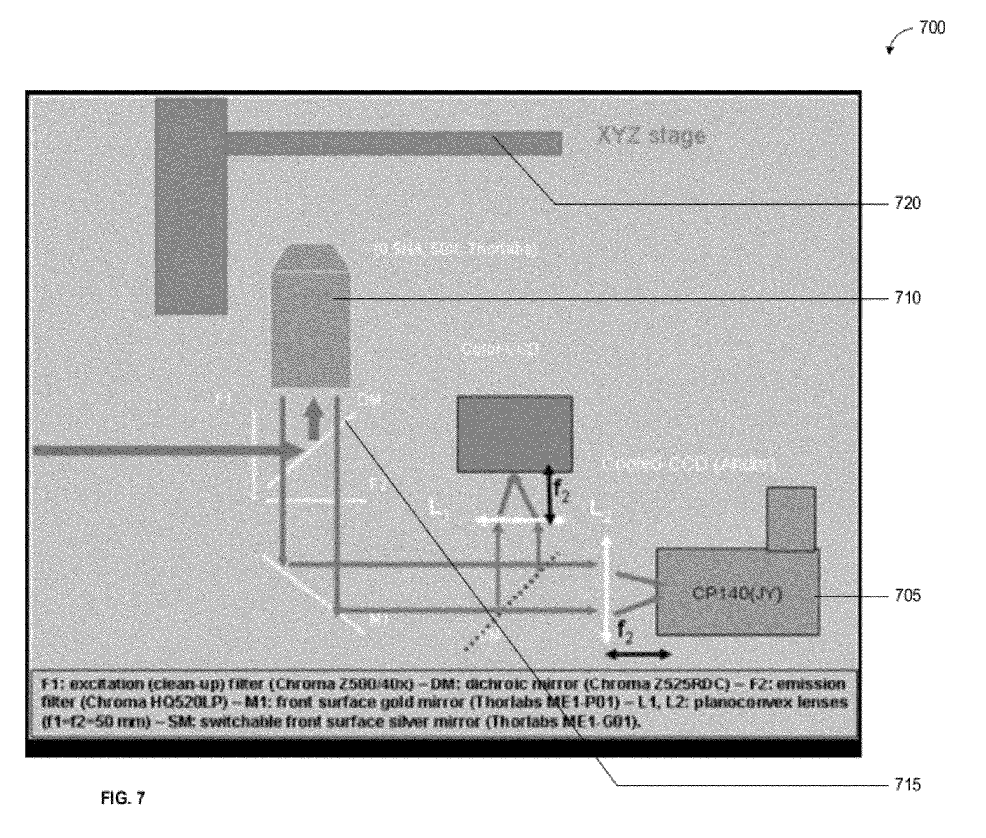

FIG. 7 depicts a schematic of an LIF detection system 700. The LIF detection system 700 can be used as or included in the detection unit 214. The LIF detection system 700 can include an argon-ion laser (488 nm and 514 nm wavelength lines) 705 which can be focused within a micro-channel of the microfluidic device. In some implementations, the micro-channel, where the laser beam is focused, can be between 40 μm and 50 μm in diameter. A microscope objective 710 with high numerical aperture, e.g., Olympus, LUCPLN FLN, 20×, NA=0.45, can be used. A dichroic mirror 715, or beam splitter, e.g., Chroma Z525RDC) can be used to direct the reflected light onto the sample via the objective and prevent the laser light reflected by the sample from further propagating into the detector. The dichroic mirror 715 will allow the fluorescent signal obtained from the DNA-dye complex to be transmitted. In some implementations, the LIF detection system 700 can include an XYZ stage on which the microfluidic device can be positioned for translation in the XYZ directions.

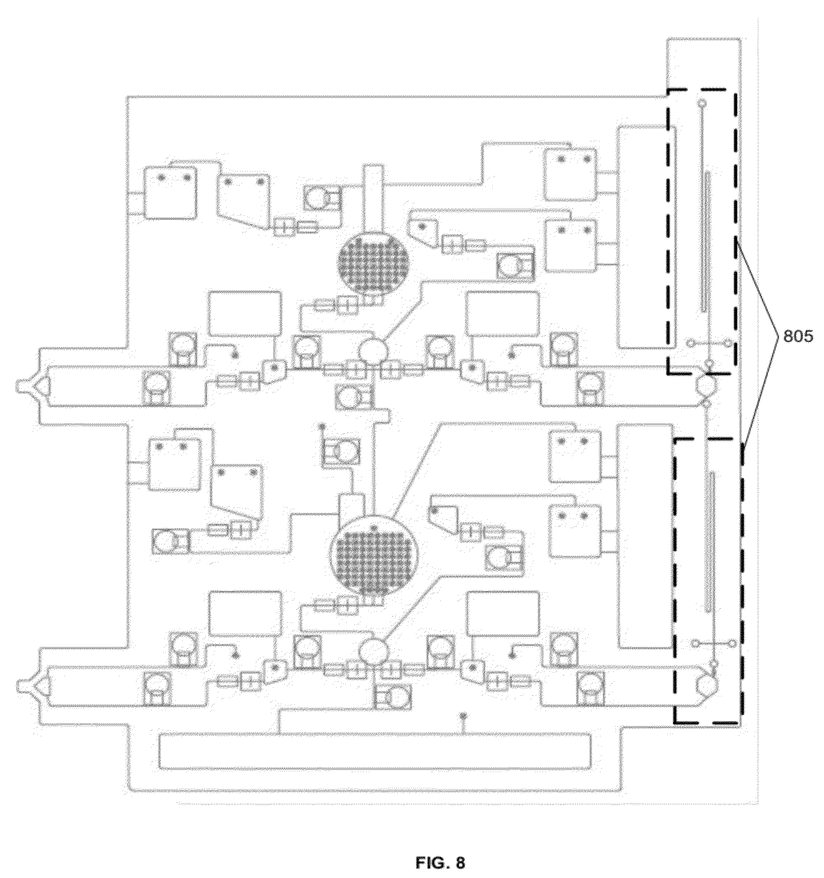

FIG. 8 depicts a schematic of a microfluidic device. The microfluidic device includes separation micro-channels 805 incorporated into the design of the microfluidic device to perform electrophoretic separation of the DNA that is transported subsequent to PCR from the amplification unit 210 to the separation unit 212.

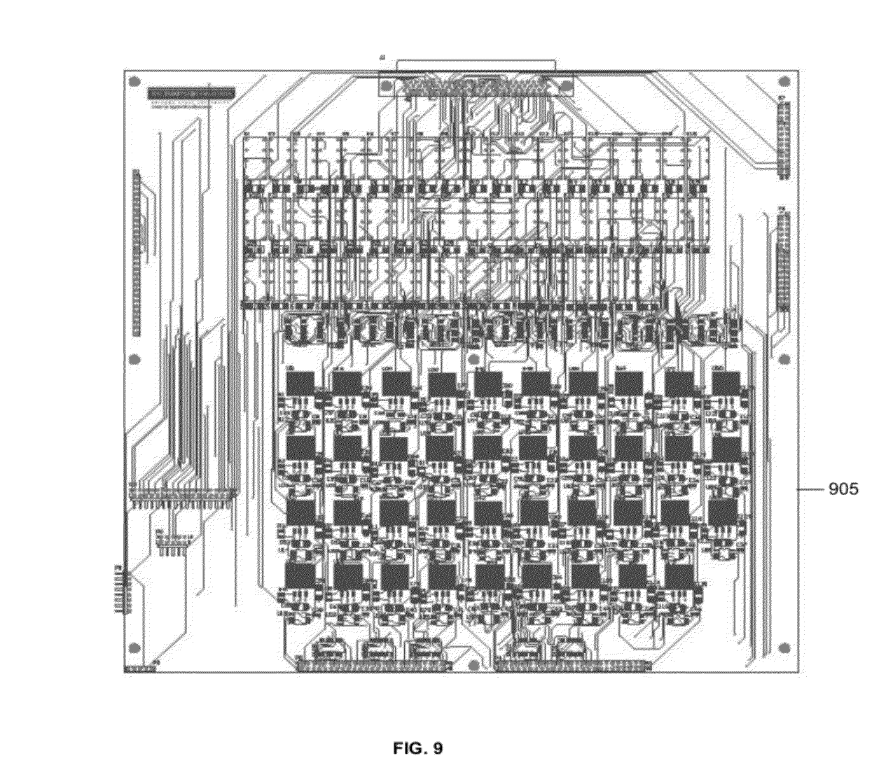

FIG. 9 depicts a schematic of an example of a printed circuit board (PCB) 905 designed to operate the peripheral devices 216 including the mechanical units 218, power sources 220, electronic units 222, and the like that are incorporated into the design of the microfluidic device 200. The PCB 905 and the microfluidic device 200 can be designed such that when microfluidic device 200 can be positioned over the PCB 905. Such positioning can cause the peripheral devices 216 on the microfluidic device 200 to be aligned with portions of the PCB 205 that operate the peripheral devices 216. The PCB 905 can also be designed such that power can be supplied to the PCB 905 from an external power source. The power supplied to the PCB 905 can be used to operate the peripheral devices 216 on the microfluidic device.

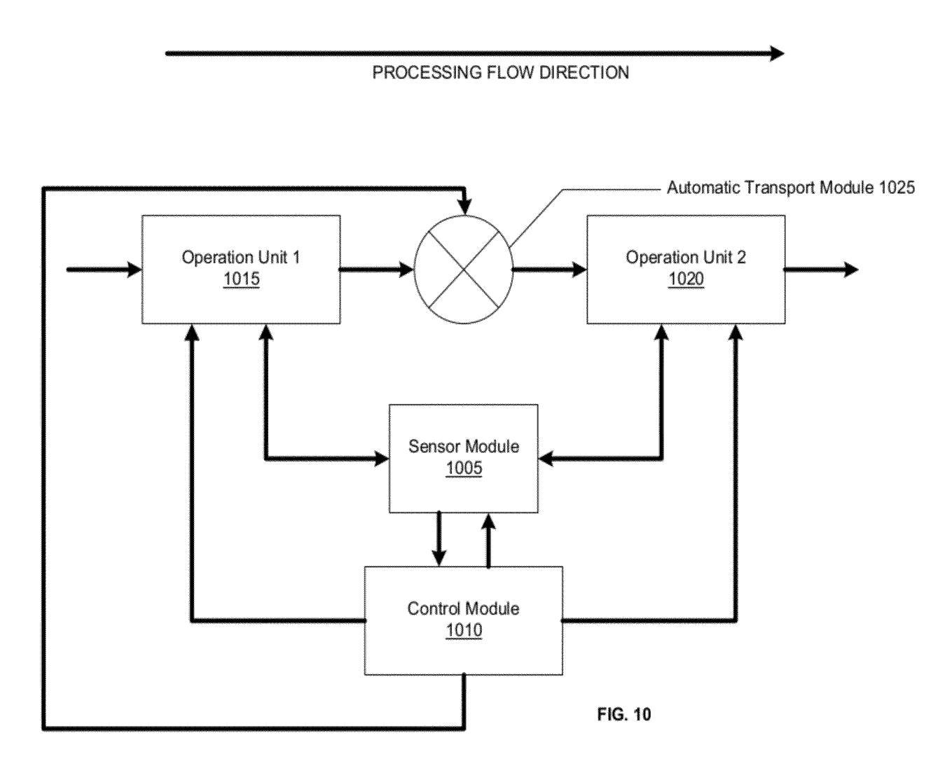

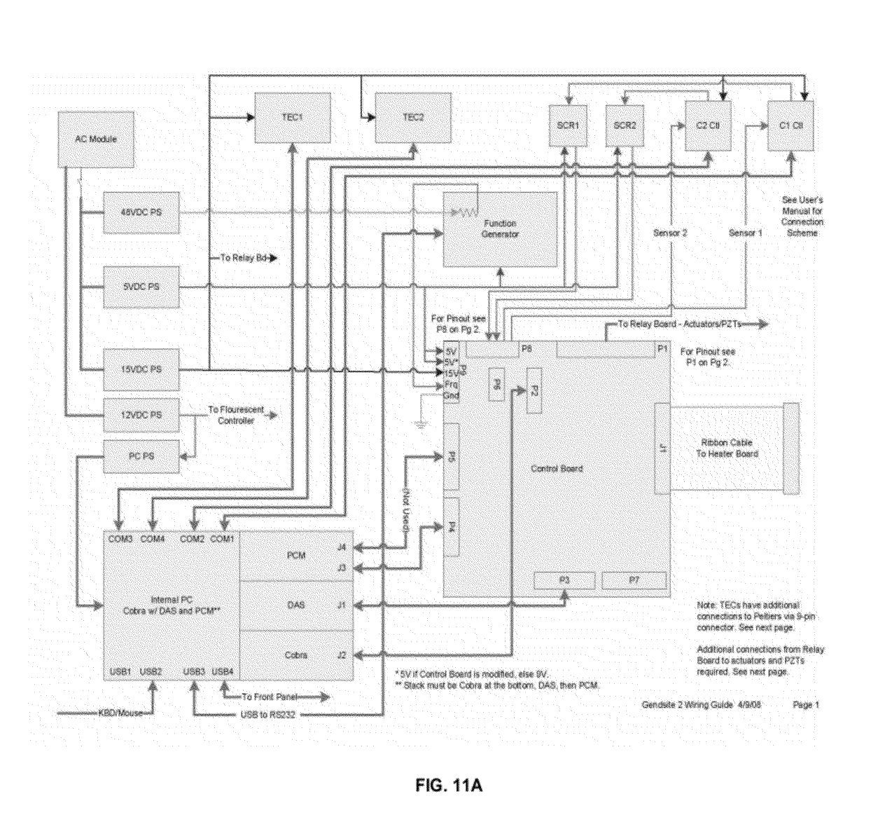

FIG. 10 depicts a schematic of an example of a sensor module to enable operation of the microfluidic device. In some implementations, the PCB 905 can include a sensor module 1005 and an control module 1010. The sensor module 1005 can be operatively coupled to operation unit 1 1015 and operation unit 2 1020 which can be a unit formed in the microfluidic device, e.g., the sample collection chamber 202, the lysis unit 206, the amplification unit 208, the amplification unit 210, the separation unit 212, and the like. In some implementations, a sample containing DNA can be processed first at operation unit 1 1015 and, subsequently, at operation unit 2 1020. For example, the operation unit 1 1015 can be the lysis unit where the sample containing DNA can be lysed to access the DNA. Subsequently, the lysed sample can be transported to operation unit 2 1020 to purify the DNA. The operation unit 1 1015 and the operation unit 2 1020 can be operatively coupled, e.g., be connected by micro-channels formed in the microfluidic device. In addition, the microfluidic device can include peripheral devices 216, e.g., micro-valves, micro-pumps, actuators, micro-mixers, and the like, to enable the operations of operation unit 1 1015 and operation unit 2 1020 as well as to transport the sample, the DNA, or both, from operation unit 1 1015 to operation unit 2 1020.

In some implementations, the sample can be received by operation unit 1 1015. The sensor module 1005 can be configured to sense the receipt of the sample at operation unit 1 1015, e.g., using a trigger formed in the microfluidic device. For example, when the sample with the DNA is received in operation unit 1 1015, a circuit may be completed and a signal can be triggered and transmitted to the sensor module 1005. Alternatively, or in addition, a change in the temperature, pressure, or both, within operation unit 1 1015 upon receiving the sample can cause the signal to be triggered and transmitted to the sensor module 1005. Upon sensing the trigger signal, the sensor module 1005 can be configured to cause the control module 1010 to transmit a signal to the peripheral devices operating operation unit 1 1015 to commence operation. In some implementations, the microfluidic device 1025 can include an automatic transport module 1025 that can receive the signal from the control module 1010 to automatically transport the sample from operation unit 1 to operation unit 2. For example, operation unit 1 1015 can be the lysis unit 206 and operation unit 2 1020 can be the purification unit 208. When the lysis unit receives the sample from the sample collection chamber 202, a trigger signal can be transmitted to the sensor module 1005 to indicate the receipt of the sample. The sensor module 1005 can cause the control module 1010 to transmit a signal to components on the microfluidic device, including the lysis unit 204 and the peripheral devices that enable the lysis unit 204 to operate. For example, the control module 1010 can transmit input signal, e.g., a voltage to operate the peripheral devices, to transport any reagents required for lysis to the lysis unit 206.

A second trigger signal can be generated upon completion of lysis in the lysis unit 204 and transmitted to the sensor module 1005. Upon receiving the second trigger signal, the sensor module 1005 can recognize the completion of the operation of the lysis unit 204 and instruct the control module 1010 to transmit trigger signals to cause the transport of the lysed sample to operation unit 2 1020, e.g., the purifying unit 208. The trigger signals transmitted to the purifying unit 208 can be, e.g., voltage inputs, to the automatic transport module 1025 including the peripheral devices that cause the operation of the purifying unit 208. In this manner, each operating unit on the microfluidic device can transmit a signal to the sensor module 1005 upon receiving sample. The sensor module 1005, in turn, can transmit trigger signals to initiate the operation of the unit from which the signal was received. Upon completing the operations, the operation unit can transmit a signal to the sensor module 1005 which can cause the control module 1010 to transport the processed sample to the subsequent operation unit on the microfluidic device and commence the operations in the subsequent operation unit. The same sensor module 1005 and control module 1010 can be configured to operate all the operation units on the microfluidic device. Alternatively, each operation unit can be associated with a corresponding sensor module and each sensor module can be configured to be in communication with other sensor modules.

In other implementations, the operations on the microfluidic device can be timing based, where input signals from the PCB 905 can be transmitted to the microfluidic device to commence operations at pre-determined times. For example, the time to transport a known volume of sample from the sample collection chamber 202 to the lysis unit 206 can be previously determined. Thus, the PCB 905 can be designed to transmit signals to cause the operation of the lysis unit 206 at a pre-determined time. In such implementations, the use of sensors to detect the completion of a previous operation can be avoided by previously determining the time to complete an operation, the time to transport sample from one unit to another, and the like.

FIG. 11A is a schematic of an example of a power supply to supply power to the automated response system. As illustrated in FIG. 11, AC input is applied through a switch to four power supplies: 48 VDC PS, 5 VDC PS, 15 VDC PS, and 12 VDC PS. The 48 VDC PS is used to power the piezo actuator actuator or PZTs through the Frequency Generator board. The 5 VDC PS powers the Control Board, the Function Generator, and the SCRs. The 15 VDC PS powers the TECs (temperature controllers for the Peltiers) and the temperature controllers for Chamber 1 and Chamber 2. The 12 VDC PS is used to power the fluorescent display and the PC. The PC is a VersaLogic Cobra computer with onboard digital lines as well as PC-104 cards installed. One of the cards is a DAS card providing analog and digital lines. The second is a PCM card providing digital lines. The computer communicates with the four temperature controllers via serial ports and the Frequency Generator via a USB-to-Serial converter. The Control Board functions as the central control point. Temperature sensors from the chambers on the Heater Board are connected through this board to the C1 and C2 controllers. The controllers switch on and off the SCRs to keep the temperature of the chambers at their required setpoints. Relays on the Control Board activate the various heaters on the Heater Board as commanded by the digital lines from the PC. The frequency output from the Frequency Generator is applied through the Control Board to the PZTs. The commands from the PC to the solenoid actuators are passed through the Control Board. Also, 15V from the 15 VDC PS is passed through the Control Board for the Relay Board (to actuate the solenoid actuators).

As illustrated in FIG. 11B, signals from the Control Board are sent via P1 to a Relay Board. This board switches on and off inputs to the four solenoid actuators (for magnet control) and the four PZTs. Additionally, fans for the Peltier assemblies are controlled through this Relay Board. On the top of the unit, a DB9 connector feeds temperature sensor signals from the top and bottom Peltier assemblies to the TEC controllers and passes on the heating and cooling signals to the Peltiers. In summary, software commands from the PC pass through the Control Board to activate and deactivate all heaters on the Heater Board. Temperature controllers are commanded by the PC via the Control Board to control the temperature on the two thermal chambers. Magnets and PZTs are actuated via digital signals from the PC through the Control Board. The frequency for the PZTs, commanded by the PC, is applied via the Control Board and the Relay Board. The Peltiers for the PCR process are controlled by the PC via TEC controllers.

A number of implementations have been described. Nevertheless, it will be understood that various modifications may be made without departing from the spirit and scope of the specification. For example, the sample can include sperm cells and epithelial cells. The ARS 105 can separate the sperm and epithelial cells, transport the two types of cells to different chambers in the microfluidic device 200 and process each type of cells separately. In some implementations, the ARS 105 can process the cells in serial manner where the sperm cells can first be processed followed by the epithelial cells, where all processing occurs in the same chambers. In other implementations, the microfluidic device 200 can include sperm cells processing chambers and epithelial cells processing chambers, where the processing can occur in parallel. In some implementations, the ARS 105 can be configured to perform real time PCR enabling quantifying the amount of DNA at specific frequencies during each cycle. In some implementations, subsequent to quantifying the DNA by real time PCR, the ARS 105 can be configured to perform multiplex PCR for STR typing.

In some implementations, an automated on-chip sample preparation and detection method can be used for examining the genetic materials of forensic evidence for rapid human identification. The method can include providing a sexual assault sample including a mixture of sperm and epithelial cells into a microfluidic device which includes self-contained actuators that are configured to perform a continuous differential cell lysis to cause the DNA of the mixed cells to be selectively extracted in different analytes. The method can be configured to direct the fluid flow of said DNA containing analytes to spatially separate microfluidic containers with on-board reagents suitable for at least 10-plex enzymatic amplification of the respective nucleic acids. Further, the DNA reaction products can be in situ labeled with at least five distinct optical color reagents, used in human loci measurements. Further, all DNA can be collected for fluid flow injection into a separation unit that can be configured for the electrophoretic migration of DNA that causes the separation of the DNA into discrete samples. A stimulating light can be impinged on the discrete samples to cause the samples to emit multiple fluorescent light signals. The emitted light signals can be detected and a set of electrophoresis data can be constructed. The data can be analyzed with a searchable database that can compare the data set with a database for rapid identification of at least a human male and female.

The on-chip sample preparation and detection method can be performed automatically using an apparatus with an integrated micro-system architecture that can include the following components: a device composed of a first polymeric substrate layer that can be processed to form some 3D features, e.g., a cavity, a micro-channel, a hole, a pillar, whose at least one dimension of said feature is <1 μm, a second polymeric substrate layer that can be attached to said first layer to preferably form a monolithic structure, a third polymeric substrate onto which electronic circuitry can be processed or assembled; an instrument comprised of a first piece of hardware that can interface with the device, and provide all necessary electronic controlling features and a second piece of software for the data acquisition and data analysis that may be needed for the examination of the genetic materials and human identification. The micro-system architecture can include microfluidic components such as channels and chambers with self-contained mechanical actuators for fluid flow manipulation such as valves, pumps, mixers, and the like. The hardware of the instrument can include electronics configured to activate and/or control actuators and/or transducers and/or sensor; a power source comprising high energy density storage and a high power density converter; the software comprised algorithms such as peak de-convolution, statistical analysis (e.g. regression, principal component analysis or PCA, PLS), image analysis, data mining or any algorithm used in human identification, an information system for tracking, handling, communicating said data representative of said genetic analysis.

Nucleic acid can be extracted and separated from a sample comprising at least two cell types wherein nucleic acids from at least two of the cell types are separated wherein the extraction and separation takes place continuously in a combined structural pattern of a microfluidic channel, a reaction chamber both connected through a fluidic connector. 6. The substrate, with which the microfluidic device is made, can exhibit physico-chemical properties compatible for interrogating the physico-chemical properties of the DNA in the sample. The polymeric substrate can be fabricated by the following steps: selecting at least one polymer material having physico-chemical properties compatible with one dimensional feature forming of dimension greater than 1 nm and 3D dimensions with aspect ratio >10-5; defining a pattern of at least a fluidic (including valving and pumping) and electronic feature; transferring said pattern into said substrate using n combination of processing steps, said n is greater than 1; and completing said fabrication using an assembly process.

While this document contains many specifics, these should not be construed as limitations on the scope of an invention or of what may be claimed, but rather as descriptions of features specific to particular embodiments of the invention. Certain features that are described in this document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or a variation of a subcombination.

Only a few implementations are disclosed. However, it is understood that variations and enhancements of the described implementations and other implementations can be made based on what is described and illustrated.

What is claimed is:

1. A method for analysis of a biological sample comprising:
receiving the sample at a sample collection chamber, wherein the sample includes deoxyribonucleic acid (DNA);
automatically transporting the sample to a lysis unit;
automatically operating the lysis unit to cause lysis to obtain access to the DNA;
automatically transporting the DNA to a purification unit;
automatically operating the purification unit to isolate the DNA from other components in the sample;
automatically transporting the isolated DNA to an amplification unit;
automatically operating the amplification unit to amplify the DNA;
automatically transporting fragments of the amplified DNA to a separation unit;
automatically operating the separation unit to separate the DNA into fragments;
automatically operating a detection unit to detect the fragments using laser induced fluorescence;
generating a profile of the DNA in the received sample based on the detecting;
comparing the generated profile with profiles of DNA stored in a database; and
upon determining that the generated profile matches one of the stored profiles, identifying the source from which the stored profile was obtained,
wherein the sample collection chamber, the lysing unit, the purifying unit, the amplification unit, and the detection unit are formed in a substrate of a microfluidic device.

2. The method of claim 1 wherein the sample is automatically loaded in the sample collection chamber.

3. The method of claim 1 wherein transporting the sample to the lysis unit comprises transporting the sample via a micro-channel formed on the microfluidic device.

4. The method of claim 1 wherein operating the lysis unit comprises:
automatically transporting lysis reagents to the lysis unit; and
automatically performing mechanical and electrical operations to cause lysis.

5. A method for analysis of a biological sample comprising:
receiving the sample, wherein the sample includes deoxyribonucleic acid (DNA);
lysing the sample to obtain access to the DNA included in the sample;
purifying the DNA in the sample to isolate the DNA from other components in the sample;
amplifying the DNA;
separating fragments of the amplified DNA;
detecting the separated fragments using laser induced fluorescence;
based on the detecting, generating a profile of the DNA in the received sample;
comparing the generated profile with profiles of DNA stored in a database; and
upon determining that the generated profile matches one of the stored profiles, identifying the source from which the stored profile was obtained,
wherein the receiving, lysing, purifying, amplifying, and detecting are performed on corresponding portions of a microfluidic device, and
wherein transporting the sample and the DNA to the portions of the microfluidic device and enabling the lysing, purifying, amplifying, separating, detecting, generating, comparing, and identifying are performed automatically without user interaction.

6. The method of claim 5 wherein receiving the sample comprises manually loading the sample.

7. The method of claim 5 wherein receiving the sample comprises automatically loading the sample.

8. The method of claim 5 wherein the microfluidic device comprises one or more chambers to store reagents for the lysing, the purifying, the amplifying, the separating, and the detecting.

9. The method of claim 8 wherein enabling the lysing, purifying, amplifying, separating, detecting, generating, comparing, and identifying are performed automatically without user interaction comprises automatically transporting the stored reagents from the one or more chambers to the corresponding portions of the microfluidic device.

10. The method of claim 5 further comprising transporting the sample from one chamber to another chamber on the microfluidic device.

11. The method of claim 5 further comprising transporting the DNA from one chamber to another chamber on the microfluidic device.

12. The method of claim 11 further comprising transporting the DNA after amplifying the DNA and before separating fragments of the amplified DNA through a valve, the valve configured to remain in a closed state during the amplifying and switch to an open state during transporting the sample.

13. The method of claim 12 wherein the valve is made of a hydrogel.

14. The method of claim 13 wherein the DNA is electrophoretically transported through the hydrogel valve.

15. The method of claim 5 wherein the sample includes sperm cells and epithelial cells.

16. The method of claim 15 wherein the sperm cells and epithelial cells are separated from one another.

17. The method of claim 16 wherein the separated sperm cells are transported to a first chamber on the microfluidic device.

18. The method of claim 16 wherein the separated epithelial cells are transported to a second chamber on the microfluidic device.

19. The method of claim 5 further comprising:
presenting a user interface including one or more menus, wherein the one or more menus represent one or more corresponding operations, the one or more operations comprising at least one of the lysing, the purifying, the amplifying, the generating, the comparing, and the identifying; and
enabling a user to provide input to the operations through the user interface.

20. The method of claim 19 wherein one of the one or more menus are displayed when the corresponding operation is performed.

21. The method of claim 20 wherein a first menu corresponding to a first operation is hidden and a second menu corresponding to a second operation is displayed when the first operation ends and the second operation begins.

22. The method of claim 19 wherein a menu displays default operating conditions for an operation.

23. The method of claim 22 wherein the default operating conditions are altered based on user input.

24. The method of claim 19 wherein the input includes operating conditions.

25. A system for biological sample analysis comprising:
a sample collection chamber formed in a substrate, the sample collection chamber having an input port to receive a sample, wherein the sample includes deoxyribonucleic acid (DNA);
a first transport mechanism configured to transport the sample from the sample collection chamber;
a lysis unit, formed in the substrate, configured to obtain access to the DNA included in the sample received from the sample collection chamber;
a second transport mechanism configured to transport the DNA from the lysis unit;
a purification unit, formed in the substrate, configured to purify the DNA in the sample to isolate the DNA from other components in the sample received from the lysis unit;
a third transport mechanism configured to transport the purified DNA from the purification unit;
an amplification unit, formed in the substrate, configured to amplify the DNA received from the purification unit;
a fourth transport mechanism configured to transport the amplified DNA;
a separation unit, formed in the substrate, configured to separate fragments of the amplified DNA received from the amplification unit;
a detection unit configured to detect the separated fragments using laser induced fluorescence, wherein a profile of the DNA in the received sample is generated based on the separated fragments detected by the detection unit; and
a profile database configured to store profiles of DNA against which the generated profile is compared,
wherein the source from which the stored profile was obtained is determined based on the comparing, and
wherein the sample collection chamber, the lysis unit, the purification unit, the amplification unit, and the separation unit are configured to operate automatically without user interaction.

26. The system of claim 25 wherein the sample is manually loaded in the sample collection chamber.

27. The system of claim 25 wherein the microfluidic device includes peripheral devices incorporated in the substrate to enable operations performed in the sample collection chamber, the lysis unit, the purification unit, the amplification unit, and the separation unit and to enable transporting the sample and the DNA from one chamber on the microfluidic device to another chamber on the microfluidic device.

28. The system of claim 25 wherein the microfluidic device comprises one or more chambers to store reagents used by the sample collection chamber, the lysis unit, the purification unit, the amplification unit, and the separation unit.

29. The system of claim 28 wherein the operating the lysis unit, the purification unit, the amplification unit, and the separation unit without user interaction comprises:
transporting the sample and the DNA to corresponding units on the microfluidic device without user interaction; and
transporting the stored reagents from the one or more chambers to the corresponding units without user interaction.

30. The system of claim 25 wherein the microfluidic device includes micro-channels to transport the sample to and from the sample collection chamber.

31. The system of claim 25 wherein the microfluidic device includes micro-channels to transport the DNA from one unit to another unit.

32. The system of claim 31 further comprising a valve configured to enable transporting the DNA from the amplification unit to the separation unit, the valve configured to remain in a closed state during the amplifying and switch to an open state during transporting the sample.

33. The system of claim 32 wherein the valve is made of a hydrogel.

34. The system of claim 33 wherein the hydrogel is positioned in a micro-channel between the amplification unit and the separation unit, and wherein a potential difference is applied across the micro-channel to cause the movement of DNA from the amplification unit to the separation unit through the hydrogel.

35. The system of claim 25 wherein the sample includes sperm cells and epithelial cells.

36. The system of claim 35 wherein the sperm cells and epithelial cells are separated from one another.

37. The system of claim 36 wherein the microfluidic device includes a first chamber into which the separated sperm cells are transported.

38. The system of claim 36 wherein the microfluidic device includes a second chamber into which the separated epithelial cells are transported.

39. A system for biological sample analysis comprising:
means for receiving the sample, wherein the sample includes deoxyribonucleic acid (DNA);
means for automatically transporting the sample from the means for receiving;
means for lysing the sample received from the means for receiving to obtain access to the DNA included in the sample;
means for automatically transporting the sample from the means for lysing;
means for purifying the DNA in the sample received from the means for lysing to isolate the DNA from other components in the sample;
means for automatically transporting the sample from the means for purifying;
means for amplifying the DNA received from the means for purifying;
means for automatically transporting the amplified DNA from the means for amplifying;
means for separating fragments of the amplified DNA received from the means for amplifying;
means for detecting the separated fragments using laser induced fluorescence;
means for generating a profile of the DNA in the received sample, based on the detecting;
means for comparing the generated profile with profiles of DNA stored in a database; and
means for identifying the source from which the stored profile was obtained, upon determining that the generated profile matches one of the stored profiles,
wherein the means for receiving, means for lysing, means for purifying, means for amplifying, and means for detecting are formed in a substrate on a microfluidic device.

40. The system of claim 39 wherein means for receiving the sample comprises means for manually loading the sample.

41. The system of claim 39 wherein the microfluidic device further comprising means for enabling operations performed in the means for receiving, means for lysing, means for purifying, means for amplifying, and means for separating and to enable transporting the sample and the DNA from one chamber on the microfluidic device to another chamber on the microfluidic device.

42. The system of claim 39 wherein the microfluidic device comprises one or more means for storing reagents for the means for lysing, the means for purifying, the means for amplifying, the means for separating, and the means for detecting.
